(19) United States
(12) Patent Application Publication
(10) Pub. No.: US 2019/0172565 A1
(43) Pub. Date: Jun. 6, 2019

(54) Proximity Based Interrogation of Portable Health Monitoring Device

(71) Applicant: International Business Machines Corporation, Armonk, NY (US)

(72) Inventors: Uri Kartoun, Cambridge, MA (US); Fang Lu, Billerica, MA (US); Kenney Ng, Arlington, MA (US); Yoonyoung Park, Cambridge, MA (US)

(21) Appl. No.: 15/832,977

(22) Filed: Dec. 6, 2017

(51) Int. Cl. G16H 10/60; G16H 40/67; A61B 5/00

(52) U.S. Cl. CPC G16H 10/60; A61B 5/6887; G16H 40/67

(57) ABSTRACT

Mechanisms are provided to implement a proximity based interrogation system. The proximity based interrogation system, transmits a short-range interrogation signal using a short-range transmission protocol, and receives a responsive communication from a portable health monitoring device associated with a patient, in response to the portable health monitoring device receiving the short-range interrogation signal. The proximity based interrogation system retrieves health monitoring information from the portable health monitoring device and correlates it with patient electronic medical record (EMR) information to generate correlated health information. The proximity based interrogation system evaluates the correlated health information to identify relevant change information in at least one of the patient's medical condition, patient's lifestyle, or patient's adherence to a previously prescribed treatment, and outputs the identified relevant change information to a client computing device that presents the relevant change information to a user.

### BACKGROUND

The present application relates generally to an improved data processing apparatus and method and more specifically to mechanisms for providing proximity based interrogation of a portable health monitoring device to assist medical personnel in evaluation of the changes in a patient's health conditions, as well as patient habits, since a last encounter with the patient.

Various types of wearable technology have recently become popular to assist people in monitoring their health, tracking their activity, tracking movements, communicating with others, and a host of other applications. One of the more popular technologies is the Fitbit™, available from Fitbit Inc., which is an activity tracker that provides a wireless enabled wearable technology for monitoring the number of steps walked, the wearer's heart rate, quality of sleep, steps climbed, and other personal metrics involved in general fitness of the wearer.

Other types of wearable technology are also available that perform similar monitoring functions, but with the wearable device having different configurations. For example, the Motiv Ring™ from Motiv Inc., provides a fitness tracker that tracks movements, heart rate, and sleep, but the wearable technology takes the form of a ring that the wearer can wear on a finger as opposed to the wristband form of the Fitbit™.

In addition, portable health monitoring technology may also take the form of various applications that may run on a portable computing device, such as a smart phone or the like, and which take advantage of the built-in sensors of the portable computing device to perform various health and/or activity monitoring operations. For example, applications such as Android Sleep App provides an application functionality for execution on an Android smart phone to monitor the sleep of a user. The application iCare Health Monitor provides blood pressure and heart rate monitoring. The application Fooducate provides a nutrition application that also allows for tracking food intake, exercise, and other variables. The application MyTrainer Dasi is a health tracking application that recommends diet and exercise routine for the user. A plethora of other applications are available for various portable computing devices to assist users in tracking their overall health.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising at least one processor and at least one memory, the at least one memory comprising instructions executed by the at least one processor to cause the at least one processor to implement a proximity based interrogation system. The method comprises transmitting, by the proximity based interrogation system, a short-range interrogation signal using a short-range transmission protocol, and receiving, by the proximity based interrogation system, a responsive communication from a portable health monitoring device associated with a patient, in response to the portable health monitoring device receiving the short-range interrogation signal. The method also comprises retrieving, by the proximity based interrogation system, health monitoring information from the portable health monitoring device in response to receiving the responsive communication. Moreover, the method comprises correlating, by the proximity based interrogation system, the health monitoring information with patient electronic medical record (EMR) information to generate correlated health information, and evaluating, by the proximity based interrogation system, the correlated health information to identify relevant change information in at least one of the patient's medical condition, patient's lifestyle, or patient's adherence to a previously prescribed treatment. Furthermore, the method comprises outputting, by the proximity based interrogation system, the identified relevant change information to a client computing device that presents the relevant change information to a user. Thus, the relevant change information that may be of importance to encountering a patient may be determined automatically responsive to the patient's portable health monitoring device coming into the short range of the short-range interrogation signal.

In some illustrative embodiments, outputting of the relevant change information to the client computing device is performed prior to the user encountering the patient. Moreover, in some illustrative embodiments, the portable health monitoring device is a portable computing device executing one or more health monitoring applications, and retrieving health monitoring information from the portable health monitoring device comprises retrieving the health monitoring information from the one or more health monitoring applications. Thus, the illustrative embodiments are able to operate in conjunction with various applications executed on various portable computing devices.

In some illustrative embodiments, the patient EMR information specifies at least one of a medical condition for which the patient is being treated, a previously prescribed treatment for the patient, or a reason for a patient's currently scheduled encounter with the user. Moreover, correlating the health monitoring information with the patient EMR information may comprise determining for the medical condition, previously prescribed treatment, or reason for the patient's currently scheduled encounter, criteria for evaluating the medical condition, previously prescribed treatment, or reason for the patient's currently scheduled encounter, based on a cognitive evaluation of the patient EMR information based on medical knowledge resources. In addition, correlating the health monitoring information with the patient EMR information may comprise selecting health monitoring information that is retrieved from the portable health monitoring device that corresponds to the criteria for evaluating the medical condition, previously prescribed treatment, or reason for the patient's currently scheduled encounter. Furthermore, retrieving health monitoring information from the portable health monitoring device in response to receiving the responsive communication may comprise sending a request to the portable health monitoring device to provide the selected health monitoring information that corresponds to the criteria.

In some illustrative embodiments, outputting the relevant change information to the client computing device that presents the relevant change information to a user comprises outputting the relevant change information as a physician interface in which the change information is prioritized in the physician interface according to a measure of relevance of the change information to the medical conditions of the patient, the treatments previously prescribed to the patient, and the reason for the patient's currently scheduled encounter.

In some illustrative embodiments, the method further comprises categorizing the relevant change information into categories of qualitative assessment of factors affecting at least one of a medical condition of the patient, a previously prescribed treatment of the patient, or a reason for a currently scheduled encounter with the patient. Moreover, outputting the relevant change information to the client computing device may comprise outputting the relevant change information as a physician interface in which the categories of the relevant change information are output.

In some illustrative embodiments, the method retrieves, by the proximity based interrogation system, health monitoring information from the portable health monitoring device in response to receiving the responsive communication at least by retrieving health monitoring information permissions associated with the patient in response to receiving the responsive communication, and retrieving a portion of available health monitoring information from the portable health monitoring device, for which the patient has granted associated health monitoring information permissions for retrieving the portion of available health monitoring information.

In some illustrative embodiments, the portable health monitoring device is one of a wearable technology device, a computerized implant implanted in the patient, a human ingestible technology device, or a smartphone executing at least one health monitoring application.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment. For example, in some illustrative embodiments, a computer program product comprising a computer readable storage medium having a computer readable program stored therein is provided, where the computer readable program, when executed on a computing device, causes the computing device to implement a proximity based interrogation system. The computer readable program causes the proximity based interrogation system to operate to transmit a short-range interrogation signal using a short-range transmission protocol, receive a responsive communication from a portable health monitoring device associated with a patient, in response to the portable health monitoring device receiving the short-range interrogation signal, and retrieve health monitoring information from the portable health monitoring device in response to receiving the responsive communication. Moreover, the computer readable program further causes the proximity based interrogation system to correlate the health monitoring information with patient electronic medical record (EMR) information to generate correlated health information, evaluate the correlated health information to identify relevant change information in at least one of the patient's medical condition, patient's lifestyle, or patient's adherence to a previously prescribed treatment, and output the identified relevant change information to a client computing device that presents the relevant change information to a user.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment. For example, in one illustrative embodiment, an apparatus is provided that comprises a processor and a memory coupled to the processor, where the memory comprises instructions which, when executed by the processor, cause the processor to implement a proximity based interrogation system. The instructions executed by the one or more processors further cause the proximity based interrogation system to operate to transmit a short-range interrogation signal using a short-range transmission protocol, receive a responsive communication from a portable health monitoring device associated with a patient, in response to the portable health monitoring device receiving the short-range interrogation signal, and retrieve health monitoring information from the portable health monitoring device in response to receiving the responsive communication. The instructions executed by the one or more processors further cause the proximity based interrogation system to correlate the health monitoring information with patient electronic medical record (EMR) information to generate correlated health information, evaluate the correlated health information to identify relevant change information in at least one of the patient's medical condition, patient's lifestyle, or patient's adherence to a previously prescribed treatment, and output the identified relevant change information to a client computing device that presents the relevant change information to a user.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example block diagram illustrating the primary operational elements of a proximity based interrogation computing system and its interaction with other computing devices in accordance with one illustrative embodiment;

FIG. 2 is an example diagram illustrating a distributed data processing environment in which aspects of the illustrative embodiments may be implemented;

FIG. 3 is a block diagram of an example data processing system in which aspects of the illustrative embodiments are implemented;

FIG. 4 is an example diagram illustrating an interaction of elements of a health services computing system associated with a proximity based interrogation system in accordance with one illustrative embodiment; and

FIG. 5 is a flowchart outlining an example operation of a proximity based interrogation system in accordance with one illustrative embodiment.

### DETAILED DESCRIPTION

Often the treatment of a patient's chronic condition requires modifications to a patient's lifestyle, which may not be generally reflected in the patient's electronic medical record (EMR) in an up-to-date manner, as it requires proactive reporting by the patient or a dialog between the patient and the physician seeing the patient. Thus, prior to seeing the patient as part of a visit or “encounter”, the physician may not have the most up-to-date information about the patient's current lifestyle situation, e.g., changes in the patient's health condition, changes in the patient's habits (e.g., exercise or activity, nutrition, sleep habits, taking of medications, etc.), and the like. Only after interacting with the patient during the visit does the physician ascertain changes in the patient's health condition, lifestyle, habits, etc. and may then have to modify their treatment of the patient based on the now identified changes, which the physician must now record in the patient's EMR in a manual manner.

It would be beneficial to have an automated mechanism for selectively interrogating, based on proximity, a patient's portable health monitoring devices to collect information about the patient's lifestyle and present that information to the physician prior to the physician meeting with the patient. Moreover, it would be beneficial to have a mechanism for automatically collecting such information from a patient's portable health monitoring devices and storing the collected information in the patient's electronic medical records rather than requiring the patient to proactively report such information and/or the physician manually record the information in the patient's electronic medical record. Thus, rather than requiring the physician to react to new information provided by the patient during the visit, it would be beneficial to have a system that actively collects and stores such information from portable health monitoring devices prior to the visit, when the patient is within a given vicinity of the physician's facilities, such that the physician may review that information prior to the visit and take into consideration such information when engaging with the patient and determining appropriate treatment for the patient. In addition, it would be beneficial to have a mechanism that cognitively evaluates such information collected from the portable health monitoring devices of the patient with regard to conditions the patient is being treated for, the particular treatments previously prescribed to the patient, and the reasons for the patient's current visit or encounter, so as to accentuate or highlight the most important changes in the patient's health, habits, and adherence to treatments for ease of review by the physician, which in turn will improve the physician's ability to address the patient's medical concerns.

The present invention provides mechanisms for selectively interrogating a patient's portable health monitoring device or devices, e.g., a FitBit™, health tracking application on a portable device such as smartphone, smart capsule such as described in U.S. Pat. No. 9,107,806, or the like, when the device comes into proximity with a proximity based interrogating system (also referred to herein simply as an “interrogation system”), such as an interrogation system installed at a physician's office, a hospital (e.g., in an emergency room department, admissions department, etc.), emergency clinic, pharmacy, medical laboratory, or the like. This information may be correlated with the patient's existing patient electronic medical records (EMRs) stored, or otherwise accessible by, the computer system with which the interrogation system is associated, e.g., the physician practice computer system. The particular information obtained from the portable health monitoring device may be correlated with criteria associated with medical conditions for which the patient is currently being treated (as indicated in the patient EMR) or for which the patient has indicated the purpose of their visit with the physician, e.g., if the patient has been treated for diabetes and is coming in for an annual check, then the portable health monitoring device may be interrogated to gather nutrition and diet information from the patient, blood glucose level information, activity information, blood pressure measurement information, etc. Moreover, the particular information may be obtained from the portable health monitoring device based on the particular previously prescribed treatments for the patient.

Information from medical knowledge resources, e.g., treatment guidelines, drug reference texts, etc., EMR data from remotely located source computing systems, and the like, may be used to perform cognitive evaluations in patient information gathered from the portable health monitoring devices so that the results of such evaluations and the most relevant information that the physician should review may be presented to the physician prior to encountering the patient during a current visit or encounter. Such cognitive evaluations may comprise natural language processing being applied to the patient EMR and/or medical knowledge resources to thereby extract semantic meaning from the content of the patient EMR and/or medical knowledge resources which may then be applied to identify criteria for evaluating the patient's medical conditions, prescribed treatments, and/or reasons for a currently scheduled encounter with the physician or other medical personnel.

Changes in patient information, as indicated by the information obtained from the portable health monitoring device, since a last visit or encounter may be automatically identified. This changed information may be prioritized based on the types of information that are most pertinent to the previously identified medical conditions of the patient, the treatments previously prescribed, and/or the reason for the patient's current visit or encounter with the physician. In this way, the most important information and changes in information may be brought to the attention of the physician who typically views the patient's records prior to encountering the patient as part of the visit. Moreover, the presentation of the most important information and changes in information may prompt the physician as to the areas where the physician may wish to gather more information directly from the patient, e.g., thresholds or the like may be established for various types of information and when the values or changes in values of this information meet or exceed such thresholds, the manner by which the corresponding values and changes are output to the physician may be modified to focus the physician's attention on these portions of information.

Moreover, the patient may control which information is automatically accessible by the physician's computing system via such proximity based interrogation. That is, the patient may specify, for each type of information collected by the portable health monitoring device(s), which information the patient agrees to automatically share with the interrogation system and which information the patient does not what to share with the interrogation system and would rather control access to themselves by requiring personal interaction with the physician or manual reporting by the patient. Such permissions may be stored in the portable health monitoring devices themselves and may be used to control responses to interrogation signals from the interrogation system, may be stored in the interrogation system based on previous configuration, and/or may be stored in a remotely located patient data source based on a registration of the patient with the remotely located patient data source.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As noted above, the present invention provides mechanisms for providing proximity based interrogation of one or more portable health monitoring devices to assist medical personnel in evaluation of the changes in a patient's health conditions, as well as patient habits, adherence to previously prescribed treatments, and the like, since a last encounter with the patient. Moreover, the present invention provides mechanisms for evaluating such changes and presenting to the medical personnel the relevant changes for the particular medical conditions of the patient, the treatments prescribed for the patient, and the reason for the patient's current visit or encounter. Such information may indicate habits of the patient with regard to taking medication, performing activities, sleep habits, and other habit factors that may affect the patient's medical conditions and treatments. Such information is generally referred to herein as lifestyle information, which is representative of the way in which the patient conducts their life and the activities that they perform on a routine basis.

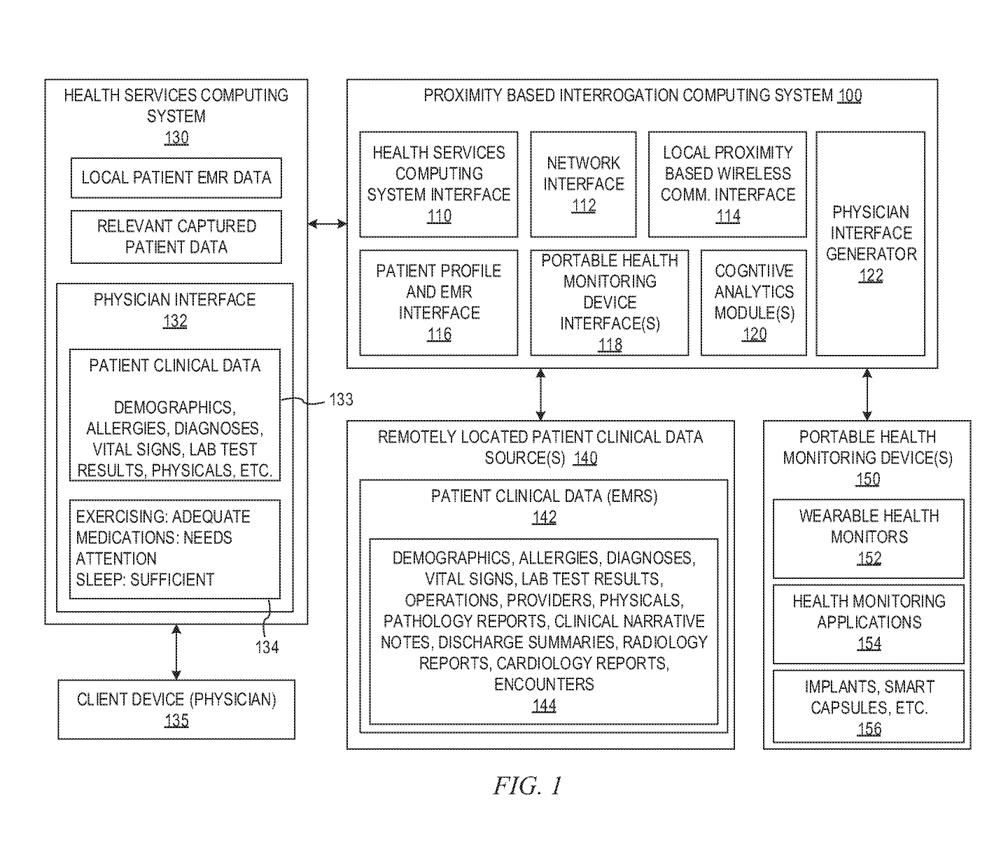

FIG. 1 is an example block diagram of a proximity based interrogation computing system in accordance with one illustrative embodiment. As shown in FIG. 1, the proximity based interrogation computing system 100 comprises a health services computing system interface 110, a network interface 112, a local proximity based wireless communication interface 114, a patient profile and electronic medical record (EMR) interface 116, one or more portable health monitoring device interfaces 118, one or more cognitive analytics modules 120, and physician interface generator 122.

The proximity based interrogation computing system 100 interfaces with a health services computing system 130 via the health services computing system interface 110. The health services computing system 130 may be any computing system associated with a provider of health services, such as a hospital, physician's office, pharmacy, clinic, laboratory, medical imaging facility, or the like. The healthcare services computing system 130 stores or has access to patient EMR data that is specific to the healthcare services computing system 130 stored locally, and/or remotely located patient EMR and clinical data 142, such as may be accessed via the proximity based interrogation computing system 100 or through a network interface and one or more data networks, from a remotely located computing device 140. The health services computing system 130 may be itself a computer that a physician accesses to obtain information about the patient, or may be a server or other computing device that provides data to client computing devices 135, such as portable tablet computing devices, workstations, or the like, that the physician may use to access the patient information. For example, the healthcare services computing system 130 may be a server located in a healthcare facility, such as a hospital, physician's office, clinic, or the like, and the physician or other healthcare worker may utilize a client computer 135 in an exam room, a portable tablet computing device 135, or the like, that has a communication connection with the healthcare services computing system 130 to access patient information from the server for display on the client computer, e.g., the physician interface 132 which will be described hereafter.

In some illustrative embodiments, the health services computing system 130 may interface with a remotely located patient clinical data source 140 via the proximity based interrogation computing system 100 and one or more data networks (not shown in FIG. 1) to obtain patient clinical data information, which may be collected from one or more other clinical data sources (not shown) and/or collected and stored from the patient's portable health monitoring device(s) 150. That is, the proximity based interrogation computing system 100 may access information stored in the remotely located patient clinical data source 140 via the network interface 112 and provide the retrieved patient information, or a designated portion thereof to which the patient has granted access to the health service computing system 130, to the health service computing system 130. Alternatively, the interrogation computing system 100 may perform one or more cognitive analytics evaluations, via cognitive analytics module(s) 120, on the retrieved information and provide the results of such cognitive analytics to the health service computing system 130.

The remotely located patient clinical data source 140 may be a cloud computing system comprising a plurality of computing device that share the responsibility for maintaining and protecting patient medical information, such as may be provided in one or more patient EMR data structures, obtained from one or more patient information source computing systems, such as via one or more data networks. Moreover, the remotely located patient clinical data source 140 may obtain patient information collected by the proximity based interrogation computing system 100 from the patient's portable health monitoring device(s) 150.

The remotely located patient clinical data source 140 may store a variety of different types of clinical data obtained from a variety of different clinical data sources. For example, the patient clinical data that may be collected and stored in the remotely located patient clinical data source 140 may include, for each of a plurality of patients, demographic information, allergy information, diagnosis information, vital sign information, laboratory test results information, medical procedure (operations) information, health services provider information and health insurance provider information, information regarding physical exams, pathology reports, clinical narrative notes, hospital/clinical discharge summary information, radiology reports, cardiology reports, and other patient encounter information.

The proximity based interrogation computing system 100 performs operations, via the portable health monitoring device interfaces 118, to interrogate the portable health monitoring device(s) 150 associated with patients to collect information stored in the portable health monitoring device(s) 150 and/or to identify the patients so that the identity of the patient may be used to retrieve stored information associated with the patient from the remotely located clinical data source 140 and/or retrieve corresponding patient EMR data stored in the health service computing system 130. The portable health monitoring device(s) 150 may take many different forms including currently known, or later developed, portable health monitoring devices. For example, these portable health monitoring devices 150 may comprise wearable health and/or activity monitoring devices 152, health and/or activity monitoring applications 154 executed on portable computing devices, such as a smart phone, tablet computing device, or other portable computing device, as well as computerized implants, smart capsules or other human ingestible technology devices, and the like 156. It should be appreciated that a single patient may have one or multiple ones of these types of portable health monitoring devices.

The portable health monitoring device interfaces 118 may comprise a plurality of different interfaces for communicating with different types of portable health monitoring devices 150 that are supported by the proximity based interrogation computing system 100. The interfaces 118 may send interrogation signals or messages, via the local proximity based wireless communication interface 114, having appropriate wireless transceiver devices (not shown), for detecting the presence of portable health monitoring devices 150 within a local proximity of the proximity based interrogation computing system 100, as well as interrogating or requesting the transfer of patient information from those detected portable health monitoring devices 150. Thus, the interfaces 118 may continuously or periodically transmit interrogation signals or messages and look for responses from any portable health monitoring devices that may be within the local proximity of the proximity based interrogation computing system 100, with initiation of a communication session with the portable health monitoring devices that respond to the interrogation signal so as to request and receive patient information from the portable health monitoring devices in accordance with the permissions established by the patient.

The interrogation signal or message sent out by the interface 118 may request that the portable health monitoring device 150 respond with the patient identifier if the portable health monitoring device 150 is within the local proximity. One of the initial operations performed in response to receiving a response signal or message from a portable health monitoring device 150 including the patient identifier, is to perform a lookup operation in the health services computing system 130 of the patient EMR data, patient scheduling information (not shown), e.g., information about the patient's appointment and the reason for the appointment or visit. Moreover, a session may be initiated via the network interface 112, with a remotely located patient clinical data source 140 to retrieve various patient clinical data and EMR data 142 which may be stored remotely, such as clinical data and EMR data 142 that may be obtained from other sources than the health services computing system 130, e.g., medical laboratories, other hospitals or physician's offices, medical imaging facilities, pharmacies, and the like.

The proximity based interrogation computing system 100, in addition to providing logic for interfacing with the various computing devices 130, 140, and 150 to obtain and provide data for facilitating operations of the various computing devices 130-150 and the operations of the proximity based interrogation computing system 100, provides additional cognitive analytics module(s) logic 120 for performing cognitive analytics based processing of the patient information collected from the portable health monitoring device(s) 150, the remotely located patient clinical data source 140, and/or the patient EMR data stored in the health service computing system 130 to generate information indicative of changes in a patient's health condition, lifestyle behaviors (habits), adherence to treatments, and the like, relevant to previous medical conditions and treatments associated with the patient, a reason for a current encounter with the physician, or the like. The cognitive analytics module(s) 120 evaluate the received patient information (patient EMR data, clinical data, portable health monitor device captured information, and the like), relative to previous medical conditions being treated, the particular treatments prescribed, and the reason for the patient's current visit (as determined from the retrieval of scheduling information from the health services computing system 130). The cognitive analytics module(s) 120 may apply medical knowledge, treatment guidelines, and the like, that may be provided in electronic documents of a corpus or otherwise stored electronically for application to patient information. Thus, the cognitive analytics module(s) 120 may implement natural language processing technology to process the patient EMR data, the various received patient information, and the medical knowledge, treatment guidelines, and the like, obtained from the corpus to extract semantic meaning and insights which may then be applied to the task of identifying relevant correlations of patient information to thereby identify relevant change information. The application of the knowledge from these medical resources to the patient information by the cognitive analytics module(s) 120 provides an indication of the most relevant change information in the patient's medical condition, patient's lifestyle information (e.g., habits), and adherence to previously prescribed treatments.

The results generated by the analytics modules 120 may be provided to the physician interface generator 122 which generates a physician interface 132 in which the relevant clinical data of the patient 133 as well as the relevant cognitive analytics module evaluation results 134 for changes in the patient's medical condition, patient's lifestyle (e.g., habits), and adherence to treatments may be provided in an accentuated manner. In the depicted example, the relevant cognitive analytics module evaluation results 134 represents an evaluation and categorization of the patient's change in habits since a last visit or encounter with the patient by the physician. In particular, the habits evaluated include exercise habits, habits with regard to taking prescribed medications, and habits with regard to sleep. These habits are evaluated based on the patient information collected from the patient's portable health monitoring device(s) 150. Thus, for example, the activity tracking capabilities, sleep monitoring capabilities, and medication reminder capabilities of the portable health monitoring devices 150 may be used to generate patient information describing the patient's activity, sleep, and responsiveness to medication reminders which can then be evaluated by the cognitive analytics modules 120 to determine if the patient is achieving a desired level of activity, getting good sleep, and is taking their medication.

The level to which the patient is achieving these goals may be categorized into different categories indicative of a need for attention by the physical and can be presented to the physician via the physician interface 132 in an accentuated manner based on the level of need for attention. For example, in the depiction of FIG. 1, the particular patient's exercise habit is adequate, their sleep is sufficient, but their taking of prescribed medications needs attention. The taking of prescribed medications habit information may be accentuated in the output of the physician interface 132 so that the physician's attention is brought to that issue requiring further inquiry by the physician.

It should be appreciated that while the physician interface generator 122 is shown as part of the proximity based interrogation computing system 100, the illustrative embodiments are not limited to such. Rather, in some embodiments, this generator 122 may be provided in the health services computing system 130 and may receive the results generated by the cognitive analytics modules 120.

The physician interface 132 may be provided to a client device 135 for output such that a physician, or other medical personnel, may access the interface 132 and view the most relevant information for the patient prior to encountering the patient. As noted above, the information output in the physician interface may be specifically customized the particular patient's previous medical conditions currently being treated, such as may be indicated in the patient's previous EMR data from a previous visit or encounter, the previously prescribed treatments that the patient should be adhering to for the medical conditions, as well as the reason for the patient's current visit or encounter. Hence, the output of the physician interface provides information to the physician that is relevant to the current visit or encounter between the physician and the patient and includes information gathered and evaluated automatically which would otherwise only be gathered after the physician had encountered the patient. The presentation of the evaluation results and the relevant clinical data is such that the most important aspects of the patient's medical condition, changes in lifestyle, and adherence to treatments are accentuated for further inquiry by the physician when treating the patient, thereby assisting with decision support operations and/or interacting with the patient during a patient encounter.

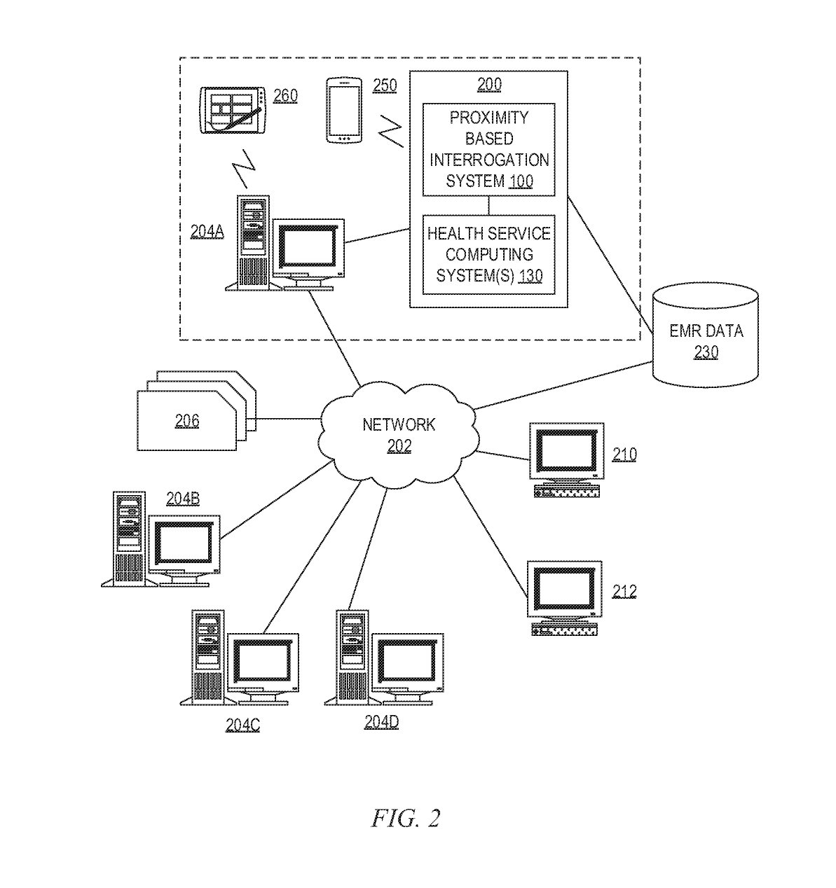

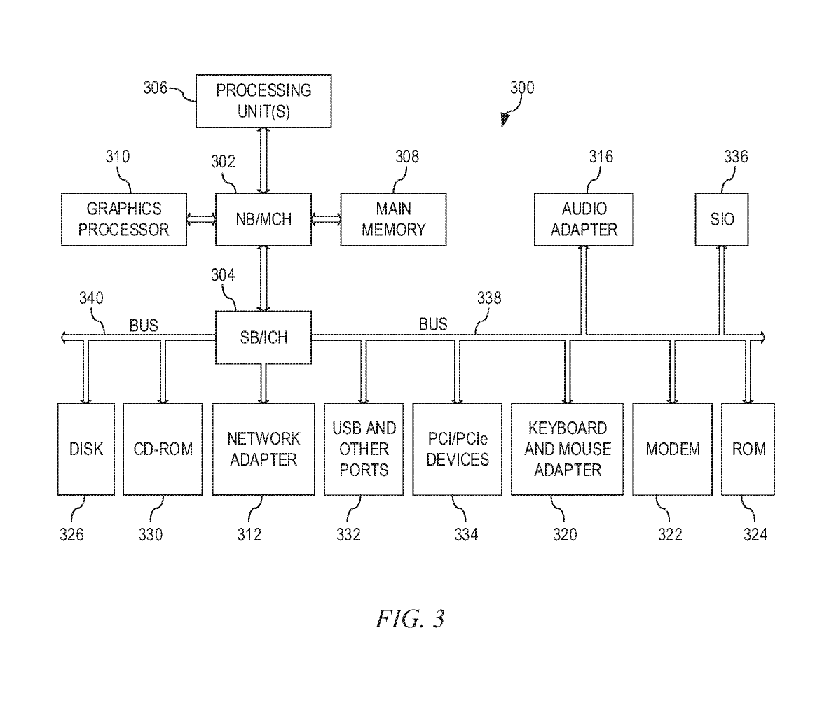

The illustrative embodiments may be utilized in many different types of data processing environments. In order to provide a context for the description of the specific elements and functionality of the illustrative embodiments, FIGS. 2-3 are provided hereafter as example environments in which aspects of the illustrative embodiments may be implemented. It should be appreciated that FIGS. 2-3 are only examples and are not intended to assert or imply any limitation with regard to the environments in which aspects or embodiments of the present invention may be implemented. Many modifications to the depicted environments may be made without departing from the spirit and scope of the present invention.

FIGS. 2-3 are directed to describing an example cognitive system for healthcare applications which implements a request processing apparatus, request processing methodology, and request processing computer program product with which the mechanisms of the illustrative embodiments are implemented. In particular, the cognitive system may comprise a proximity based interrogation system 220 and a health services computing system 200 which operate in conjunction to interrogate portable health monitoring devices of patients and provide cognitive evaluations of the patient information gathered from such interrogations, and from other sources, to evaluate changes in a patient's medical condition, lifestyle behavior (e.g., habits), and adherence to treatments, since a last visit or encounter between the physician and the patient. This functionality is performed in response to detecting the presence of the portable health monitoring device(s) of the patient within a local proximity 270 of the proximity based interrogation system 220 and prior to the physician encountering the patient.

The proximity based interrogation system 220 may implement one or more cognitive analytics modules, such as cognitive analytics modules 120 in FIG. 1, which themselves may utilize request processing pipelines to perform evaluations of patient information with regard to the particular configured analytics that that module 120 is to perform. It should be appreciated that e cognitive analytics module 120 may utilize a request processing pipeline that is separately trained and/or configured to process patient information associated with different domains. For example, in some cases, a first request processing pipeline of a first analytics module may be trained to operate on patient information with regard to a first medical malady domain (e.g., various types of blood diseases) while another request processing pipeline may be trained to operate on patient information with regard to another medical malady domain (e.g., various types of cancers). Moreover, each request processing pipeline of each of the cognitive analytics modules 120 may have their own associated corpus or corpora of medical knowledge, treatment guidelines, and the like, that they ingest and operate on, e.g., one corpus for blood disease domain documents and another corpus for cancer diagnostics domain related documents in the above examples.

As an overview, a cognitive system, or in this case a cognitive analytics module 120, is a specialized module configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware.

IBM Watson™ is an example of one such cognitive system which can process human readable language and identify inferences between text passages with human-like high accuracy at speeds far faster than human beings and on a larger scale. The cognitive system, e.g., IBM Watson™, may be used to provide cognitive analytics modules 120 which are configured specifically to perform analytics evaluations on patient information gathered from the portable health monitoring devices 150, the health services computing system 130, remotely located patient clinical data sources 140, and other medical resource sources of one or more corpora 206 in FIG. 2. The cognitive systems of the cognitive analytics modules 120 may generate change information indicating the most relevant changes in patient medical condition, patient lifestyle, and treatment adherence for use by the physician when encountering the patient.

As shown in FIG. 2, the cognitive health system 200, comprising the proximity based interrogation system 100 and the health services computing system(s) 130, is implemented on one or more computing devices 204A-D (comprising one or more processors and one or more memories, and potentially any other computing device elements generally known in the art including buses, storage devices, communication interfaces, and the like) connected to the computer network 202. For purposes of illustration only, FIG. 2 depicts the cognitive health system 200 being implemented on computing device 204A only, but as noted above the cognitive health system 200 may be distributed across multiple computing devices, such as a plurality of computing devices 204A-D. The network 202 includes multiple computing devices 204A-D, which may operate as server computing devices, and client computing devices 210-212, in communication with each other and with other devices or components via one or more wired and/or wireless data communication links, where each communication link comprises one or more of wires, routers, switches, transmitters, receivers, or the like.

In some illustrative embodiments, the cognitive health system 200 and network 202 enable proximity based interrogation of portable health monitoring devices 250 when such devices enter a local proximity region 270 of the proximity based interrogation system 100, as previously described above with regard to FIG. 1. As described previously, the proximity based interrogation system 100 may also operate on stored EMR data and clinical data for the patient 230, which may be locally stored or remotely stored on remotely located computing devices accessible via network 202), as well as medical knowledge obtained from medical knowledge resources, such as may be provided in one or more corpora 206. For example, the cognitive health system 200 may access a corpus or corpora of electronic documents 206 via the network 202, where portions of the corpus or corpora 206 may be provided on one or more server computing devices, network attached storage devices, in one or more databases, or other computing devices not explicitly shown in FIG. 2. The network 202 includes local network connections and remote connections in various embodiments, such that the cognitive health system 200 may operate in environments of any size, including local and global, e.g., the Internet.

The electronic documents of the one or more corpora 206 may include any file, text, article, or source of data for use in the cognitive health system 200 and may be provided in a structured or unstructured manner, e.g., natural language documents which may be processed using natural language processing to extract medical knowledge, treatment characteristics, and the like. For example, the electronic documents of the one or more corpora 206 may comprise medical knowledge bases, medical condition diagnosis knowledge sources, treatment guidelines, and the like, that specify various knowledge, criteria, and characteristics of medical conditions and treatments for such medical conditions. This information may be correlated with the medical conditions and treatments associated with the patient. This information may be used to determine what information to retrieve from the portable health monitoring devices 250. The EMR data and patient information collected from the portable health monitoring devices 150 may further be evaluated based on this knowledge obtained from the one or more corpora 206 to determine what criteria are met or not met, what certain ranges of values may represent, what portions of treatments are satisfied and what consequences may occur based on a failure to satisfy certain portions of the treatments, etc. This information may be used to identify relevant changes in patient medical condition, patient lifestyle (e.g., habits), and treatment adherence that are to be accentuated in a physician interface, as discussed previously.

The physician interface may be provided to, or otherwise accessed, by a physician or other medical personnel via a client computing device local to the local proximity 270, for example. In the depicted example, the client computing device is a portable tablet type computing device 260 which has graphical display capabilities used to provide a graphical user interface output of the physician interface. The output on the client device 260 may comprise patient clinical data and relevant change information for the patient medical condition, lifestyle, and treatment adherence. The physical interface displays the information gathered and evaluated by the cognitive analytics modules of the proximity based interrogation system 100 to identify the relevant changes for output to the physician prior the physician encountering the patient. The cognitive analytics modules may apply thresholds, logic functions, and the like, to determine a degree or level of relevance or importance which may be used to identify an accentuation or particular representation of the change information to be used to provide the information in the physician's interface and thereby direct the physician's attention to the change information. In this way, the most relevant change information based on the most up-to-date changes in the patient's medical condition, lifestyle, and treatment adherence as communicated from the portable health monitoring devices may be brought to the physician's attention prior to encountering the patient so that this information may be used when engaging with the patient.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 3 is provided as an example of one type of data processing system in which aspects of the present invention may be implemented. Many other types of data processing systems may be likewise configured to specifically implement the mechanisms of the illustrative embodiments.

FIG. 3 is a block diagram of an example data processing system in which aspects of the illustrative embodiments are implemented. Data processing system 300 is an example of a computer, such as server 204 or client 210 in FIG. 2, in which computer usable code or instructions implementing the processes for illustrative embodiments of the present invention are located. In one illustrative embodiment, FIG. 3 represents a server computing device, such as a server 204, which implements a cognitive system 200 that includes the mechanisms of the illustrative embodiments described herein.

In the depicted example, data processing system 300 employs a hub architecture including North Bridge and Memory Controller Hub (NB/MCH) 302 and South Bridge and Input/Output (I/O) Controller Hub (SB/ICH) 304. Processing unit 306, main memory 308, and graphics processor 310 are connected to NB/MCH 302. Graphics processor 310 is connected to NB/MCH 302 through an accelerated graphics port (AGP).

In the depicted example, local area network (LAN) adapter 312 connects to SB/ICH 304. Audio adapter 316, keyboard and mouse adapter 320, modem 322, read only memory (ROM) 324, hard disk drive (HDD) 326, CD-ROM drive 330, universal serial bus (USB) ports and other communication ports 332, and PCl/PCIe devices 334 connect to SB/ICH 304 through bus 338 and bus 340. PCl/PCIe devices may include, for example, Ethernet adapters, add-in cards, and PC cards for notebook computers. PCI uses a card bus controller, while PCIe does not. ROM 324 may be, for example, a flash basic input/output system (BIOS).

HDD 326 and CD-ROM drive 330 connect to SB/ICH 304 through bus 340. HDD 326 and CD-ROM drive 330 may use, for example, an integrated drive electronics (IDE) or serial advanced technology attachment (SATA) interface. Super I/O (SIO) device 336 is connected to SB/ICH 304.

An operating system runs on processing unit 306. The operating system coordinates and provides control of various components within the data processing system 300 in FIG. 3. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 300.

As a server, data processing system 300 may be, for example, an IBM® eServer™ System p® computer system, running the Advanced Interactive Executive) (AIX®) operating system or the LINUX® operating system. Data processing system 300 may be a symmetric multiprocessor (SMP) system including a plurality of processors in processing unit 306. Alternatively, a single processor system may be employed.

Instructions for the operating system, the object-oriented programming system, and applications or programs are located on storage devices, such as HDD 326, and are loaded into main memory 308 for execution by processing unit 306. The processes for illustrative embodiments of the present invention are performed by processing unit 306 using computer usable program code, which is located in a memory such as, for example, main memory 308, ROM 324, or in one or more peripheral devices 326 and 330, for example.

A bus system, such as bus 338 or bus 340 as shown in FIG. 3, is comprised of one or more buses. Of course, the bus system may be implemented using any type of communication fabric or architecture that provides for a transfer of data between different components or devices attached to the fabric or architecture. A communication unit, such as modem 322 or network adapter 312 of FIG. 3, includes one or more devices used to transmit and receive data. A memory may be, for example, main memory 308, ROM 324, or a cache such as found in NB/MCH 302 in FIG. 3.

Those of ordinary skill in the art will appreciate that the hardware depicted in FIGS. 2 and 3 may vary depending on the implementation. Other internal hardware or peripheral devices, such as flash memory, equivalent non-volatile memory, or optical disk drives and the like, may be used in addition to or in place of the hardware depicted in FIGS. 2 and 3. Also, the processes of the illustrative embodiments may be applied to a multiprocessor data processing system, other than the SMP system mentioned previously, without departing from the spirit and scope of the present invention.

Moreover, the data processing system 300 may take the form of any of a number of different data processing systems including client computing devices, server computing devices, a tablet computer, laptop computer, telephone or other communication device, a personal digital assistant (PDA), or the like. In some illustrative examples, data processing system 300 may be a portable computing device that is configured with flash memory to provide non-volatile memory for storing operating system files and/or user-generated data, for example. Essentially, data processing system 300 may be any known or later developed data processing system without architectural limitation.

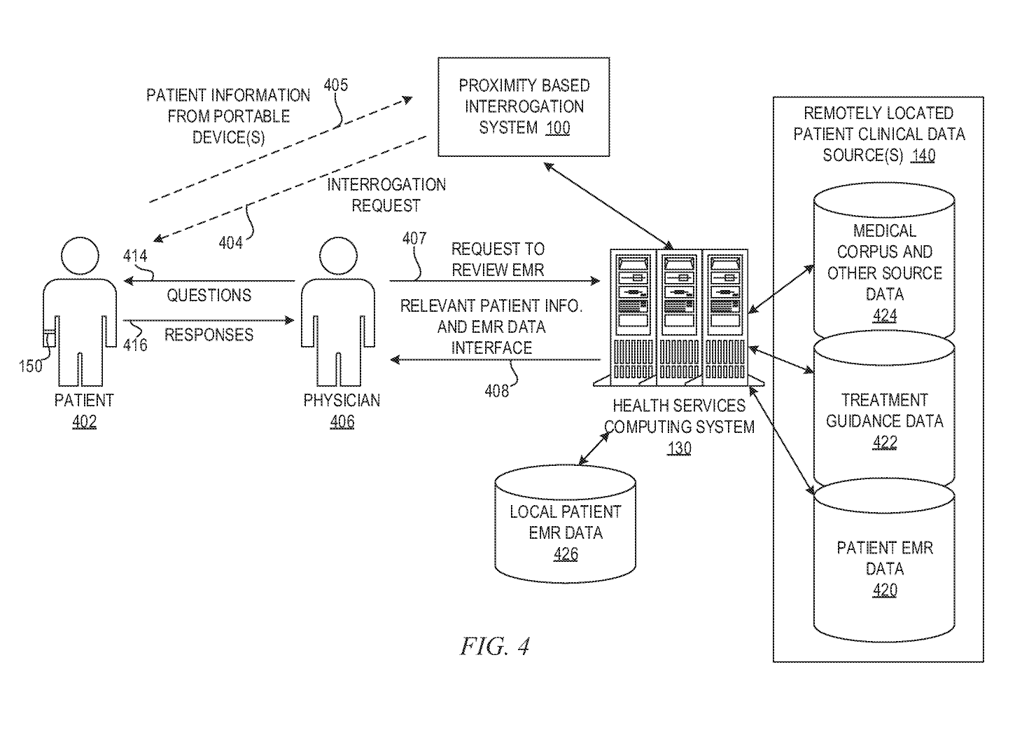

FIG. 4 is an example diagram illustrating an interaction of elements of a healthcare cognitive system in accordance with one illustrative embodiment. The example diagram of FIG. 4 depicts an implementation of a healthcare cognitive system that is configured to provide decision support services to a physician in the way of cognitive evaluation of patient information collected from portable health monitoring devices associated with patients in response to these portable health monitoring devices entering a local proximity of a proximity based interrogation system 100. The results of such evaluation may be presented to the physician 406 prior to the physician 406 encountering the patient 402, such as in an exam room. The results of the evaluation take into account the most up-to-date and relevant change information for changes in patient medical conditions, lifestyle, and adherence to prescribed treatments in an automated manner without requiring the patient to actively report such information and without requiring the physical to obtain the information from the patient through an encounter.

In response to the patient 402, having a portable health monitoring device 150, entering a local proximity of the proximity based interrogation system 100, the portable health monitoring device 150 receives the interrogation request (e.g., a signal or message) 404 from the proximity based interrogation system 100 and responds with the patient identifier in a responsive communication 405. The proximity based interrogation system 100 may lookup patient EMR data in local patient EMR data storage 426, remotely located patient EMR data storage 420, or the like, and may identify any previously specified permissions associated with the patient. Moreover, the interrogation system 100 may also identify previous medical conditions for which the patient is being treated and the particular previously prescribed treatments. This may identify what information to request from the portable health monitoring devices 150 of the patient 402.

It should be appreciated that while FIG. 4 depicts the patient 402 and user 406 as human figures, the interactions with and between these entities may be performed using computing devices, medical equipment, and/or the like, such that entities 402 and 406 may in fact be computing devices, e.g., client computing devices. For example, the interactions 414 and 416 between the patient 402 and the user 406 may be performed orally, e.g., a physician interviewing a patient, and may involve the use of one or more medical instruments, monitoring devices, or the like, to collect information that may be input to the health services computing system 130 as patient attributes 418. Interactions between the user 406 and the health services computing system 130 will be electronic via a user computing device (not shown), such as a client computing device, portable computing device, or the like, communicating with the health services computing system 130 via one or more data communication links and potentially one or more data networks.

With the mechanisms of the illustrative embodiments, the proximity based interrogation computing system 100 operates in conjunction with the health services computing system 130 associated with a health services provider facility, such as a hospital, physician's office, clinic, pharmacy, laboratory, medical imaging facility, or any other provider of health services. The interrogation system 100 is configured to include one or more portable health monitoring device interfaces 118 that are each configured to specifically communicate with one or more types of portable health monitoring devices, health monitoring applications on portable devices, smart capsules, or any other portable health monitoring mechanism 150 that may be associated with a patient 402. When a user enters the proximity of the interrogation system 100, e.g., within a WiFi area, Bluetooth area, or other limited short range communication area of the proximity based interrogation system 100, communication is made between the interrogation system 100 and the patient's portable health monitoring device(s) 150, e.g., a FitBit, health tracking application on a portable device such as smartphone, smart capsules, or the like. The interrogation system 100 obtains a patient identifier and, if the patient has provided sufficient permissions, may retrieve, directly from the portable health monitoring device 150, patient information tracked by the portable health monitoring device 150, e.g., activity information, eating habit information, sleeping pattern information, vital sign data, weight information, medication adherence information, or any other health oriented information collected and stored in the portable health monitoring device 150 for tracking various aspects of the patient's biometrics, activity, nutrition, or general health.

In some illustrative embodiments, the interrogation may involve obtaining directly from the portable health monitoring device 150 the patient identifier, with subsequent patient clinical data information being accessed from the remotely located computing system 140, such as a server or cloud based system, which has previously obtained the patient's health monitoring information from the portable health monitoring device 150 via another communication connection, e.g., a data network connection, and from other computing systems associated with other health service providers. In such illustrative embodiments, permissions information may be stored at the remote server, cloud based system, or the like, 140 which may be checked before providing such data to the health service provider computer system 130 associated with the interrogation system 100.

This information may be correlated with the patient's existing patient electronic medical records (EMRs) stored, or otherwise accessible by, the health services computer system 130 with which the interrogation system 100 is associated, such as from local patient EMR data storage 426 and/or remotely located patient EMR data storage 420. The particular information obtained from the portable health monitoring device(s) 150 may be correlated with criteria associated with medical conditions for which the patient is currently being treated (as indicated in the patient EMR data), the particular treatments prescribed, or for which the patient has indicated the purpose of their visit or encounter with the physician. Changes in such information since a last visit or encounter may be automatically identified.

For example, assume that a patient 402 has been previously seen by a physician 406 for treatment of a diabetes Type 2 medical condition. The physician 406 has previously advised the patient 402 to monitor the patient's blood pressure and blood sugar levels, lose weight, and increase activity level. The patient 402 wears a wearable health monitoring device 150 that monitors the patient's activity level, sleeping patterns, weight, heart rate, and blood pressure. The patient 402 returns to the physician 406 for a follow-up visit, where the physician's office location has an interrogation system 100 that detects the presence of the patient's wearable health monitoring device 150 (hereafter referred to simply as the “patient device”).

The interrogation system 100 communicates with the patient device 150 to obtain the identity of the patient 402. The identity of the patient 402 may then be correlated with the current appointment calendar of the health service computer system 130 (hereafter referred to simply as the “provider system”) with which the interrogation system 100 is associated, as well as the patient's EMR data. Patient consent or permission information may be retrieved, such as from the remotely located computing system 140, e.g., a centralized server or cloud based system, to determine what information is obtainable from the patient device 150 and the corresponding information may be retrieved from the patient device 150. Alternatively, the permissions may be configured in the patient device 150 itself such that the patient device 150 only divulges the information for which the patient 402 has given permission to share the information. In some embodiments, a private cloud based mechanism, such as described in commonly assigned and co-pending U.S. patent application Ser. No. 15/824,248 (Attorney Docket No. AUS920160577US1) may be utilized to protect the patient's personal health information and control dissemination of patient information from the patient device 150. The patient information reported by the patient device 150 for which the patient 402 has provided consent/permission for the patient information to be shared with the physician system 130, may be obtained by the physician system 130.

From the patient EMR data, the medical conditions and the current treatments that the patient 402 may be undergoing, may be identified. The corresponding patient information obtained indirectly from the patient device 150 via the remotely located computing system 140 or directly from the patient device 150, that is relevant to the current treatments and/or medical conditions, may be identified and correlated with previous version of this information to identify changes in the information. The most up-to-date information and indications of changes may be reflected in a notification 408 presented to the physician 406 via the physician system 130 in association with the patient's EMR data when the physician 406 is viewing this information. Similarly, a determination of the reason for the patient's visit may be identified from the appointment system (a subsystem of the physician system 130, for example), which may include an indicator of the medical condition(s) for which the patient 402 is seeking treatment. This information may be correlated with information indicative of the important patient characteristics and health information for treating the medical condition(s), as may be obtained from medical guidelines, clinical guidance from subject matter experts, and the like, which may be stored as data structures in one or more resource data sources 422, 424. This information may then be used to select health information and health information changes that are relevant to the reasons for the patient's visit which may then be identified in a notification 408 to the physician 406 via the health services computing system 130.

Such presentation of the notification to the physician 406 may be specifically in response to the physician 406 requesting 507 access to the patient's EMR data. Moreover, based on the presentation of the notification, e.g., the physician's interface, the physician 406 may review the patient's relevant change information and clinical data prior to encountering the patient 402. Thereafter, the physician 406 may then conduct an encounter with the patient 402 via questions 414 and responses 416 based on the change information and clinical data presented to the physician 406 via the notification, or physician's interface.

It should be appreciated that this identification of relevant information and changes may be with regard to lifestyle information including activity levels, eating habits (such as may be obtained from food log applications or the like), adherence to treatments, taking of medication, and the like. In this way, the most important information and changes in information may be brought to the attention of the physician 406 who typically views the patient's records prior to encountering the patient 402 as part of the visit and may prompt the physician 406 as to the areas where the physician 406 may wish to gather more information directly from the patient via the questions 414 and evaluation of patient responses 416. Moreover, as noted above, it should be appreciated that the patient 402 may control which information is automatically accessible by the health services computing system 130 via such proximity based interrogation through the setting of the permissions. When used in conjunction with a private cloud mechanism, such as described in the commonly assigned and co-pending U.S. patent application Ser. No. 15/824,248 (Attorney Docket No. AUS920160577US1), complete control over sensitive patient information is made possible.

In some embodiments, the patient's progress with regard to the reported health information from the patient device 150 may be compared to similar information for similar patients having similar demographics and/or medical conditions. Information for similar patients may be obtained from the remotely located patient clinical data sources 140 and may be used as a comparison by the cognitive analytics modules 120 of the proximity based interrogation system 100. Based on results of the comparison, modifications to the patient's treatment may be determined to attempt to bring the patient's monitored health information in conformance with other patients that are determined to be achieving success with their treatments of their medical condition. Indications of how the current patient 402 differs or is similar to other patients with regard to their changes in medical condition, lifestyle, or treatment adherence may be included in the physician's interface along with treatment options corresponding to treatments prescribed to other patients similar to the current patient 402. This information may be used by the physician 406 when determining how to modify the treatment of the patient 402 to more likely result in a successful outcome. In some embodiments, the physician can compare a set of patients who share similar demographic profiles and have gone through successful treatment with this patient. The physician could help the patient visualize what successful factors such as medication, physical activities and diet have been applied to other patients through the treatment pathway.

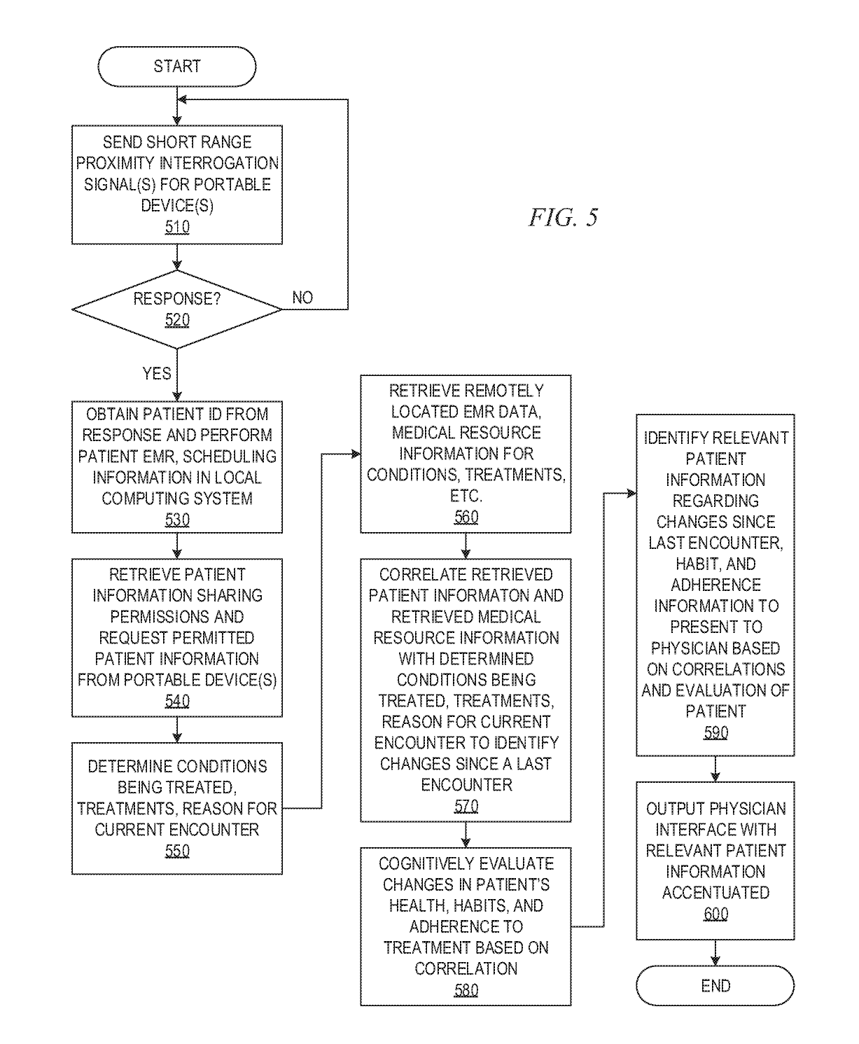

FIG. 5 is a flowchart outlining an example operation of a proximity based interrogation system in accordance with one illustrative embodiment. As shown in FIG. 5, the operation starts by sending a short ranged proximity interrogation signal or signals, e.g., WiFi signal, Bluetooth signal, IR signal, or the like, for portable health monitoring devices (step 510). A determination is made as to whether a portable health monitoring device responds to the interrogation signal (step 520). If not, the operation may return to step 510 and continue to send or transmit the interrogation signals.

In response to receiving a responsive message or signal from a portable health monitoring device, the patient identifier is obtained and a lookup operation is performed to retrieve corresponding patient EMR data and scheduling information (step 530). Moreover, information sharing permissions that have been established by the patient may be retrieved and patient information from the portable health monitoring devices is requested in accordance with the sharing permissions (step 540). The medical conditions being treated, prescribed treatments, and the reason for the current visit or encounter with the patient are determined based on the obtained patient EMR data and scheduling information (step 550). Moreover, remotely located EMR data and medical knowledge resource information for medical conditions, treatments, and the like, are also obtained (step 560). The retrieved information from the portable health monitoring devices, the local patient EMR data, and the remotely located sources (patient information) is correlated with the determined conditions being treated, prescribed treatments, and reasons for the current encounter, and compared to previous values for the patient information to identify relevant changes since a last encounter with the patient (step 570).

The changes are cognitively evaluated by cognitive analytics modules to thereby evaluate changes in the patient's health and medical condition, changes in patient lifestyle or habits, and adherence to treatments based on the correlation (step 580). The relevant patient information regarding changes since last encounter, changes in habits, and changes in the adherence to treatments, and the like, that should be presented to the physician prior to the physician encountering the patient are determined (step 590). A physician interface is generated and output with the relevant patient information and changes accentuated (step 600) and the operation terminates.

Thus, the illustrative embodiments provide mechanisms for automatically and proactively interrogating portable health monitoring devices to obtain up-to-date information regarding a patient's medical condition, lifestyle, and adherence to treatments and evaluating such prior to the physician encountering the patient. As a result, the physician is presented with accurate information upon which the physician can base an encounter with the patient and need to extract the information from the patient via oral inquiries.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and at least one memory, the at least one memory comprising instructions executed by the at least one processor to cause the at least one processor to implement a proximity based interrogation system, wherein the method comprises:
transmitting, by the proximity based interrogation system, a short-range interrogation signal using a short-range transmission protocol;
receiving, by the proximity based interrogation system, a responsive communication from a portable health monitoring device associated with a patient, in response to the portable health monitoring device receiving the short-range interrogation signal;
retrieving, by the proximity based interrogation system, health monitoring information from the portable health monitoring device in response to receiving the responsive communication;
correlating, by the proximity based interrogation system, the health monitoring information with patient electronic medical record (EMR) information to generate correlated health information;
evaluating, by the proximity based interrogation system, the correlated health information to identify relevant change information in at least one of the patient's medical condition, patient's lifestyle, or patient's adherence to a previously prescribed treatment; and
outputting, by the proximity based interrogation system, the identified relevant change information to a client computing device that presents the relevant change information to a user.

2. The method of claim 1, wherein outputting of the relevant change information to the client computing device is performed prior to the user encountering the patient.

3. The method of claim 1, wherein the portable health monitoring device is a portable computing device executing one or more health monitoring applications, and wherein retrieving health monitoring information from the portable health monitoring device comprises retrieving the health monitoring information from the one or more health monitoring applications.

4. The method of claim 1, wherein the patient EMR information specifies at least one of a medical condition for which the patient is being treated, a previously prescribed treatment for the patient, or a reason for a patient's currently scheduled encounter with the user.

5. The method of claim 4, wherein correlating the health monitoring information with the patient EMR information comprises:
determining for the medical condition, previously prescribed treatment, or reason for the patient's currently scheduled encounter, criteria for evaluating the medical condition, previously prescribed treatment, or reason for the patient's currently scheduled encounter, based on a cognitive evaluation of the patient EMR information based on medical knowledge resources; and
selecting health monitoring information that is retrieved from the portable health monitoring device that corresponds to the criteria for evaluating the medical condition, previously prescribed treatment, or reason for the patient's currently scheduled encounter.

6. The method of claim 4, wherein retrieving health monitoring information from the portable health monitoring device in response to receiving the responsive communication comprises sending a request to the portable health monitoring device to provide the selected health monitoring information that corresponds to the criteria.

7. The method of claim 1, wherein outputting the relevant change information to the client computing device that presents the relevant change information to a user comprises outputting the relevant change information as a physician interface in which the change information is prioritized in the physician interface according to a measure of relevance of the change information to the medical conditions of the patient, the treatments previously prescribed to the patient, and the reason for the patient's currently scheduled encounter.

8. The method of claim 1, further comprising categorizing the relevant change information into categories of qualitative assessment of factors affecting at least one of a medical condition of the patient, a previously prescribed treatment of the patient, or a reason for a currently scheduled encounter with the patient, and wherein outputting the relevant change information to the client computing device comprises outputting the relevant change information as a physician interface in which the categories of the relevant change information are output.

9. The method of claim 1, wherein retrieving, by the proximity based interrogation system, health monitoring information from the portable health monitoring device in response to receiving the responsive communication further comprises:
retrieving health monitoring information permissions associated with the patient in response to receiving the responsive communication; and
retrieving a portion of available health monitoring information from the portable health monitoring device, for which the patient has granted associated health monitoring information permissions for retrieving the portion of available health monitoring information.

10. The method of claim 1, wherein the portable health monitoring device is one of a wearable technology device, a computerized implant implanted in the patient, a human ingestible technology device, or a smartphone executing at least one health monitoring application.

11. A computer program product comprising a computer readable storage medium having a computer readable program stored therein, wherein the computer readable program, when executed on a computing device, causes the computing device to implement a proximity based interrogation system which operates to:
transmit a short-range interrogation signal using a short-range transmission protocol;
receive a responsive communication from a portable health monitoring device associated with a patient, in response to the portable health monitoring device receiving the short-range interrogation signal;
retrieve health monitoring information from the portable health monitoring device in response to receiving the responsive communication;
correlate the health monitoring information with patient electronic medical record (EMR) information to generate correlated health information;
evaluate the correlated health information to identify relevant change information in at least one of the patient's medical condition, patient's lifestyle, or patient's adherence to a previously prescribed treatment; and
output the identified relevant change information to a client computing device that presents the relevant change information to a user.

12. The computer program product of claim 11, wherein the computer readable program causes the proximity based interrogation system to output the relevant change information to the client computing device prior to the user encountering the patient.

13. The computer program product of claim 11, wherein the portable health monitoring device is a portable computing device executing one or more health monitoring applications, and wherein the computer readable program causes the proximity based interrogation system to retrieve health monitoring information from the portable health monitoring device at least by retrieving the health monitoring information from the one or more health monitoring applications.

14. The computer program product of claim 11, wherein the patient EMR information specifies at least one of a medical condition for which the patient is being treated, a previously prescribed treatment for the patient, or a reason for a patient's currently scheduled encounter with the user.

15. The computer program product of claim 14, wherein the computer readable program causes the proximity based interrogation system to correlate the health monitoring information with the patient EMR information at least by:
determining for the medical condition, previously prescribed treatment, or reason for the patient's currently scheduled encounter, criteria for evaluating the medical condition, previously prescribed treatment, or reason for the patient's currently scheduled encounter, based on a cognitive evaluation of the patient EMR information based on medical knowledge resources; and
selecting health monitoring information that is retrieved from the portable health monitoring device that corresponds to the criteria for evaluating the medical condition, previously prescribed treatment, or reason for the patient's currently scheduled encounter.

16. The computer program product of claim 14, wherein the computer readable program causes the proximity based interrogation system to retrieve health monitoring information from the portable health monitoring device in response to receiving the responsive communication at least by sending a request to the portable health monitoring device to provide the selected health monitoring information that corresponds to the criteria.

17. The computer program product of claim 11, wherein the computer readable program causes the proximity based interrogation system to output the relevant change information to the client computing device that presents the relevant change information to a user at least by outputting the relevant change information as a physician interface in which the change information is prioritized in the physician interface according to a measure of relevance of the change information to the medical conditions of the patient, the treatments previously prescribed to the patient, and the reason for the patient's currently scheduled encounter.

18. The computer program product of claim 11 wherein the computer readable program further causes the proximity based interrogation system to categorize the relevant change information into categories of qualitative assessment of factors affecting at least one of a medical condition of the patient, a previously prescribed treatment of the patient, or a reason for a currently scheduled encounter with the patient, and wherein the computer readable program causes the proximity based interrogation system to output the relevant change information to the client computing device at least by outputting the relevant change information as a physician interface in which the categories of the relevant change information are output.

19. The computer program product of claim 11, wherein the computer readable program causes the proximity based interrogation system to retrieve health monitoring information from the portable health monitoring device in response to receiving the responsive communication further at least by:
retrieving health monitoring information permissions associated with the patient in response to receiving the responsive communication; and
retrieving a portion of available health monitoring information from the portable health monitoring device, for which the patient has granted associated health monitoring information permissions for retrieving the portion of available health monitoring information.

20. An apparatus comprising:
a processor; and
a memory coupled to the processor, wherein the memory comprises instructions which, when executed by the processor, cause the processor to implement a proximity based interrogation system which operates to:
transmit a short-range interrogation signal using a short-range transmission protocol;
receive a responsive communication from a portable health monitoring device associated with a patient, in response to the portable health monitoring device receiving the short-range interrogation signal;
retrieve health monitoring information from the portable health monitoring device in response to receiving the responsive communication;
correlate the health monitoring information with patient electronic medical record (EMR) information to generate correlated health information;
evaluate the correlated health information to identify relevant change information in at least one of the patient's medical condition, patient's lifestyle, or patient's adherence to a previously prescribed treatment; and
output the identified relevant change information to a client computing device that presents the relevant change information to a user.
